(19) United States
(12) Patent Application Publication
(10) Pub. No.: US 2025/0381027 A1
(43) Pub. Date: Dec. 18, 2025

(54) Expandable Support Frame and Medical Device

(71) Applicant: Cook Medical Technologies LLC, Bloomington, IN (US)

(72) Inventors: Joshua Krieger, Bloomington, IN (US); Sean Chambers, Bloomington, IN (US); Zachary Berwick, Indianapolis, IN (US); Ghassan Kassab, Zionsville, IN (US)

(73) Assignee: Cook Medical Technologies LLC, Bloomington, IN (US)

(21) Appl. No.: 19/177,825

(22) Filed: Apr. 14, 2025

Related U.S. Application Data
(63) Continuation of application No. 17/883,080, filed on Aug. 8, 2022, now abandoned.
Application No. 17/883,080 is a continuation of application No. 15/804,049, filed on Nov. 6, 2017, now Pat. No. 11,406,495.
Application No. 15/804,049 is a continuation of application No. 14/176,364, filed on Feb. 10, 2014, now abandoned.
(60) Provisional application No. 61/763,107, filed on Feb. 11, 2013.

(51) Int. Cl. A61F 2/24; A61F 2/90; A61F 2/92

(52) U.S. Cl. CPC A61F 2/2418; A61F 2/90; A61F 2/92; A61F 2220/0016; A61F 2220/0058

(57) ABSTRACT

Support frames and medical devices are described. An example medical device comprises an expandable support frame with first and second leaflets attached to the support frame. Each of the first and second leaflets defines a domed radius that is equal to or less than the radius of the expandable support frame when the expandable support frame is in an expanded configuration and the leaflets are subjected to fluid pressure sufficient to affect closure of the valve orifice.

### CROSS REFERENCE TO RELATED APPLICATIONS

This application is a continuation of U.S. patent application Ser. No. 17/883,080, filed Aug. 8, 2022, which is a continuation of U.S. patent application Ser. No. 15/804,049, filed on Nov. 6, 2017, which is a continuation of U.S. patent application Ser. No. 14/176,364, filed on Feb. 10, 2014, and which claims the benefit of U.S. Provisional Application No. 61/763,107, filed on Feb. 11, 2013. The entire contents of each of these related applications is incorporated into this disclosure by reference.

### FIELD

The disclosure relates generally to the field of implantable medical devices. More particularly, the disclosure relates to intraluminal support frames and medical devices. Particular embodiments relating to intraluminal valve devices and support frames suitable for use in such devices are described in detail.

### BACKGROUND

Expandable intraluminal support frames have proven useful in the medical arts. Some expandable support frames are useful without inclusion of any additional elements. Stents, for example, are routinely used in several body lumens as a means for providing support to ailing vessels, such as coronary and non-coronary vessels. In some medical devices, an expandable support frame provides a scaffold onto which one or more additional elements can be attached to achieve a desired function. Occlusion devices, for example, often include a graft or other sheet-like material attached to an expandable support frame. Constructed in this way, these medical devices can be delivered and deployed intraluminally to substantially block fluid flow through a body vessel. Similarly, some valve devices include a leaflet or leaflets attached to an expandable support frame in a manner that allows the leaflet or leaflets to move between open and closed positions. Constructed in this way, these medical devices can be delivered and deployed intraluminally to regulate fluid flow through a body vessel.

Considering these roles of intraluminal support frames in the medical arts, a need exists for improved frames. Furthermore, for the various types of intraluminal medical devices that include a support frame and one or more additional elements, a need exists for improved frames that improve the effectiveness of the composite device.

Valve devices provide an example. Several researchers have pursued the development of prosthetic valves that are implantable by minimally invasive techniques. Indeed, the art now contains several examples of implantable venous valve devices. Many of these prior art devices include an expandable support frame and an attached graft member that is fashioned into a valve that regulates fluid flow through the device and, ultimately, a body vessel. For example, a graft member can be in the form of a leaflet that is attached to a support frame and movable between first and second positions. In a first position, the valve is open and allows fluid flow to proceed through a vessel in a first direction, and in a second position the valve is closed to prevent fluid flow in a second, opposite direction. Examples of this type of prosthetic valve are described in commonly owned U.S. Pat. No. 6,508,833 to Pavcnik for a MULTIPLE-SIDED INTRALUMINAL MEDICAL DEVICE, which is hereby incorporated by reference in its entirety.

Despite this and other examples, a need remains for improved medical devices, including implantable valve devices, that include an expandable support frame.

### BRIEF OVERVIEW OF EXAMPLE EMBODIMENTS

Various example support frames and medical devices are described and illustrated herein.

An example support frame comprises a first circumferential serpentine path; a second circumferential serpentine path; a first connector segment joining the first and second serpentine paths, the first connector segment comprising substantially parallel first and second struts; a second connector segment disposed substantially opposite the first connector segment with respect to the longitudinal axis of the support frame and joining the first and second serpentine paths, the second connector segment comprising substantially parallel third and fourth struts; a third connector segment disposed circumferentially adjacent the first and second connector segments and joining the first and second serpentine paths; a fourth connector segment disposed substantially opposite the third connector segment and joining the first and second serpentine paths; a first connector strut extending between and joining the first and third connector segments; and a second connector strut extending between and joining the second and third connector segments.

An example medical device comprises an expandable support frame having a longitudinal axis, an outer circumference, an unexpanded configuration, and an expanded configuration with an expanded configuration radius extending from the longitudinal axis to the outer circumference; a first leaflet attached to the support frame along a first attachment pathway, the first leaflet having a first inner surface that defines a domed radius equal to or less than the expanded configuration radius when the support frame is in the expanded configuration; and a second leaflet attached to the support frame along a second attachment pathway, the second leaflet having a second inner surface that defines a second domed radius equal to or less than the expanded configuration radius when the support frame is in the expanded configuration.

Another example medical device comprises an expandable support frame having a longitudinal axis, an outer circumference, an unexpanded configuration, and an expanded configuration with an expanded configuration radius extending from the longitudinal axis to the outer circumference. For this example medical device, the expandable support frame comprises a first circumferential serpentine path; a second circumferential serpentine path; a first connector segment joining the first and second serpentine paths, the first connector segment comprising substantially parallel first and second struts; a second connector segment disposed substantially opposite the first connector segment with respect to said longitudinal axis and joining the first and second serpentine paths, the second connector segment comprising substantially parallel third and fourth struts; a third connector segment disposed circumferentially adjacent the first and second connector segments and joining the first and second serpentine paths; a fourth connector segment disposed substantially opposite the third connector segment and joining the first and second serpentine paths; a first connector strut extending between and joining the first and third connector segments; and a second connector strut extending between and joining the second and third connector segments. This example medical device includes a leaflet attached to the support frame along an attachment pathway extending along the first and second connector struts and along a portion of the first connector segment and a portion of the second connector segment, the leaflet having an inner surface that defines a domed radius equal to or less than the expanded configuration radius when the support frame is in the expanded configuration. The domed radius can be any suitable domed radius, including a domed radius that is between about ⅛^{th} the expanded configuration radius and the expanded configuration radius, a domed radius that is between about ¼^{th} the expanded configuration radius and about ¾^{th} the expanded configuration radius, and a domed radius that is about ¼^{th} the expanded configuration radius.

Another example medical device is similar to the example medical device described above, but also includes a second leaflet attached to the support frame along a second attachment pathway extending along the third and fourth connector struts and along a portion of the first connector segment and a portion of the second connector segment. Similar to the first leaflet, the second leaflet can have a domed radius equal to or less than the expanded configuration radius when the support frame is in the expanded configuration. For the second leaflet, the domed radius can be any suitable domed radius, including a domed radius that is between about ⅛^{th} the expanded configuration radius and the expanded configuration radius, a domed radius that is between about ¼^{th} the expanded configuration radius and about ¾^{th} the expanded configuration radius, and a domed radius that is about ¼^{th} the expanded configuration radius.

In another example medical device having first and second leaflets, as briefly described above, the first and second leaflets have domed radii that are substantially equal. In another example medical device having first and second leaflets, as briefly summarized above, the first and second leaflets have domed radii that are equal.

Additional understanding of the inventive support frames and medical devices can be obtained with review of the detailed description, below, and the appended drawings.

### BRIEF DESCRIPTION OF THE DRAWINGS

FIG. 1 is a perspective view of a first example support frame.

FIG. 2 another perspective view of the first example support frame, rotated ninety degrees from the view illustrated in FIG. 1.

FIG. 3 is a side view of a second example support frame.

FIG. 4 is another side view of the second example support frame, rotated ninety degrees from the view illustrated in FIG. 3.

FIG. 5 is a flat plan view of the support frame illustrated in FIGS. 3 and 4.

FIG. 6 is a side view of a third example support frame.

FIG. 6A is a partial side view of an alternate support frame.

FIG. 6B is a partial side view of another alternate support frame.

FIG. 7 is another side view of the third example support frame, rotated ninety degrees from the view illustrated in FIG. 6.

FIG. 8 is a perspective view of a first example medical device.

FIG. 9 is a side view of a second example medical device.

FIG. 10 is another side view of the second example medical device, rotated ninety degrees from the view illustrated in FIG. 9.

FIG. 11 is a side view of a third example medical device.

FIG. 12 is another side view of the third example medical device, rotated ninety degrees from the view illustrated in FIG. 11.

### DETAILED DESCRIPTION OF ILLUSTRATED EXAMPLE EMBODIMENTS

The following detailed description and the appended drawings describe and illustrate various example support frames and medical devices that are embodiments of the invention. The description and drawings are exemplary in nature and are provided to enable one skilled in the art to make and use one or more support frames or medical devices as an embodiment of the invention. The description and drawings are not intended to limit the scope of the claims in any manner.

Inventive intraluminal support frames and medical devices are described. The support frames are useful in the making of intraluminal medical devices, including the medical devices described herein. The support frames may also be useful as medical devices themselves, such as intraluminal stents. The medical devices can be used in any suitable intraluminal environment and to achieve any desired treatment effect in an animal, including human and non-human animals. For example, some of the example medical devices are useful for regulating fluid flow through a body vessel of a patient. As such, the medical devices can be used as valve devices. The medical devices also may be useful for other intraluminal purposes.

### Support Frames

FIGS. 1 and 2 illustrate a first example support frame 100.

The support frame 100 is an expandable support frame comprising proximal 102 and distal 104 portions connected by various connector segments 106, 108, 110, 112. The proximal portion 102 defines a first serpentine path 114 that extends around the circumference of the support frame 100. The distal portion 104 defines a second serpentine path 116 that also extends around the circumference of the support frame 100. The first serpentine path 114 includes pairs of straight strut portions 118 and bends 120, each of which is disposed between and connected to a circumferentially adjacent pair of the connector segments 106, 108, 110, 112. The second serpentine path 116 includes curvilinear struts 122, 124, 126, 128. Similar to the first serpentine path 114, each of the curvilinear struts 122, 124, 126, 128 is disposed between and connected to a circumferentially adjacent pair of the connector segments 106, 108, 110, 112. Thus, each serpentine path 114, 116 is joined to connector segments 106, 108, 110, 112.

In the illustrated embodiment, each of the connector segments 106, 108, 110, 112 includes first and second straight struts, designated by the corresponding reference number along with a or b, e.g., 110a, 110b, that are disposed parallel to each other. For each of the connector segments 106, 108, 110, 112, the straight struts are connected to each other by to curvilinear struts, designated by the corresponding reference number along with c or d, e.g., 110c, 110d. This arrangement of struts in the connector segments 106, 108, 110, 112 is considered advantageous at least because it provides a degree of structural redundancy and gives a secondary attachment point for associated materials and/or components in medical devices that include the support frame 100. In the illustrated embodiment, each of connector segments 106, 108, 110, 112 is disposed substantially on the circumferential plane of the support frame 100. It is noted, though, that one or more of the connector segments in a support frame according to a particular embodiment can be disposed entirely or partially outside of the circumferential plane of the support frame 100. For example, one or more connector segments may include a bend or curve that projects outwardly with respect to a longitudinal axis of the support frame. Connector segments with these structural features may be advantageous when additional surface area for contact with a wall of a body vessel and/or formation of an artificial sinus is desired, for example.

The support frame 100 illustrated in FIGS. 1 and 2 has four connector segments 106, 108, 110, 112. Pairs of these connector segments are disposed substantially opposite one another with respect to a longitudinal axis a of the support frame 100. Thus, connector segments 106 and 108 are disposed substantially opposite each other with respect to longitudinal axis a, and connector segments 110 and 112 are disposed substantially opposite each other with respect to longitudinal axis a. As a result, connector segments 106, 108, 110, 112 are distributed on the circumference of the support frame 100 such that each connector segment 106, 108, 110, 112 is positioned approximately equidistantly from two other connector segments 106, 108, 110, 112 along the circumference. It is noted, though, that, in all embodiments that include more than one connector segment, the connector segments can be distributed on the circumference of the support frame in any suitable manner. The illustrated distributions are merely examples of suitable distributions.

While the support frame 100 illustrated in FIGS. 1 and 2 includes four connector segments 106, 108, 110, 112, a support frame according to a particular embodiment can include any suitable number of connector segments. A skilled artisan will be able to determine an appropriate number of connector segments for a particular support frame based on various considerations, including the nature and size of the body vessel into which the support frame, or a medical device containing the support frame, is intended to be implanted and the nature of any materials and/or additional components that will be attached to the support frame in the fabrication of a medical device. When additional rigidity is desired, a greater number of connector segments can be included. When less is desired, one, two or three connector segments can be included. Furthermore, additional or fewer connector segments can be included to accommodate other materials and/or elements of a medical device in which the support frame is used. For example, the use of one, two or three connector segments may be advantageous in valve devices in which contact between a valve leaflet and a vessel wall is desirable.

In the illustrated embodiment, each of the curvilinear struts 122, 124, 126, 128 extends between and joins two of the connector segments 106, 108, 110, 112. For example, as best illustrated in FIG. 1, curvilinear strut 122 extends between and joins connector segments 106 and 110. Specifically, curvilinear strut 122 is connected to one curvilinear strut 110d of connector segment 110 and one curvilinear strut 106d of connector segment 106. Similarly, curvilinear strut 124 extends between and joins connector segments 108 and 110. Specifically, curvilinear strut 124 is connected to one curvilinear strut 110d of connector segment 110 and one curvilinear strut 108d of connector segment 108. As best illustrated in FIG. 2, curvilinear strut 126 extends between and joins connector segments 108 and 112. Thus, curvilinear strut 126 is connected to one curvilinear strut 118d of connector segment 108 and one curvilinear strut 112d of connector segment 112. While not visible in the FIGS., curvilinear strut 128 extends between and joins connector segments 112 and 106.

Inclusion of the curvilinear struts at only the distal end 104 of the support frame 100 provides directionality to the structure of the support frame 100, which is considered advantageous at least because it facilitates fabrication of medical devices that include the support frame 100. It is noted, though, that one or more curvilinear struts can be included on the proximal end, or at any other desirable location, of a support frame according to a particular embodiment.

Each curvilinear strut 122, 124, 126, 128 can have any suitable curvilinear configuration. A skilled artisan will be able to determine an appropriate configuration for a support frame according to a particular embodiment based on various considerations, including the nature of the body vessel within which the support frame is intended to be used, and the nature, size and configuration of any materials and/or additional elements that are attached to the support frame in the fabrication of a medical device that includes the support frame. Examples of suitable curvilinear configurations include curvilinear forms that define arcs, circular arcs, great arcs, s-curves, and others. Furthermore, in any particular embodiment, each curvilinear strut, if multiple curvilinear struts are included, can have the same or different curvilinear configuration as another of the curvilinear struts in the support frame. In the illustrated example embodiment, each of the curvilinear struts has the same curvilinear configuration. While considered advantageous for this illustrated example, this is merely an example of a suitable configuration and arrangement.

The inventors have determined that curvilinear struts that define circular arcs are particularly advantageous for inclusion in the support frames described herein. For example, each of the curvilinear struts 122, 124, 126, 128 in the embodiment illustrated in FIGS. 1 through 2 defines a circular arc.

For a curvilinear strut that defines an arc that is a circular arc or great arc, the arc can comprise a segment of the circumference of any suitable circle. As a result, the arc can have any suitable radius of curvature. A skilled artisan will be able to select an appropriate radius of curvature for such an arc for a support frame according to a particular embodiment based on various considerations, such as the nature and size of the body vessel within which the support frame is to be implanted, the number of curvilinear struts included in the support frame, and the nature, size and/or configuration of any additional material or elements included in a medical device within which the support frame is used.

The inventors have determined that a radius of curvature that is based on the radius of the circumference of the support frame in its expanded configuration provides desirable structural properties. For these structural measurements, the circumference of the support frame is a circumference of a transverse cross-section of the support frame with respect to the longitudinal axis of the support frame. The radius can be measured to either an inner or an outer circumferential surface, or a hypothetical circumferential surface by extension of an actual surface, of the support frame. For example, inclusion of one or more curvilinear struts that define an arc having a radius of curvature that is between about 1/16^{th} the radius of the circumference of the support frame in its expanded configuration and about 1× the radius of the circumference of the support frame in its expanded configuration is suitable. Additional examples of suitable radii of curvature for curvilinear struts include radii of curvature between about ⅛^{th} the radius of the circumference of the support frame in its expanded configuration and about 1× the radius of the circumference of the support frame in its expanded configuration is suitable, radii of curvature between about ¼^{th} and about ¾^{th} the radius of the circumference of the support frame in its expanded configuration, and a radius that is about ½ the radius of the circumference of the support frame in its expanded configuration.

In the embodiment illustrated in FIGS. 1 and 2, the support frame 100 includes four curvilinear struts 122, 124, 126, 128, each of which defines an arc having a radius of curvature r that is about ½ the radius R of the circumference of the support frame in its expanded configuration. The inventors have determined that this configuration and number of curvilinear struts 122, 124, 126, 128 is advantageous for inclusion on support frames according to particular embodiments at least because of the beneficial structural properties provided by the arrangement. Furthermore, as described in more detail below, the inventors have determined that this configuration and number of curvilinear struts 122, 124, 126, 128 is advantageous for inclusion in medical devices according to particular embodiments at least because of the attachment pathways defined by the curvilinear struts 122, 124, 126, 128.

In the illustrated embodiment, support frame 100 includes first 140 and second 142 support struts, each of which extends between and is connected to two of the curvilinear struts 122, 124, 126, 128. While considered optional, the inclusion of support struts 140, 142 may provide desirable structural properties for support frames and/or medical devices according to particular embodiments. If included, the support struts can have any suitable size and configuration. For example, the support struts can comprise straight struts or curvilinear struts. As illustrated in FIGS. 1 and 2, the support struts 140, 142 can comprise parabolic-shaped struts. Also, if included, the support struts can extend from the respective curvilinear struts at any suitable location on each of the curvilinear struts joined by the support strut. For example, as best illustrated in FIG. 1, support strut 140 extends from a point proximal to the curve defined by each of the joined curvilinear struts 122, 124. The inventors have determined that this positioning is advantageous at least because it provides desirable structural properties while not significantly interfering with the attachment pathway defined by the support frame 100 when the support frame 100 is used within a medical device and an additional material and/or additional element is attached to the curvilinear struts 122, 124 along the attachment pathway, as described below.

In all embodiments, the support frame advantageously comprises an expandable support frame having radially compressed and radially expanded configurations. Such a support frame can be implanted at a point of treatment within a body vessel by minimally invasive techniques, such as delivery and deployment with a catheter sized and conFIG.d for navigation within the body vessel. It is noted, though, that support frames and medical devices according to embodiments of the invention, regardless of the type and/or nature of the support frame, can be implanted by other techniques, including surgical techniques.

In all embodiments, the support frame can provide a stenting function, i.e., exert a radially outward force on the interior wall of a vessel in which the support frame, or medical device including the support frame, is implanted. By including a support frame that exerts such a force, a medical device according to the invention can provide multiple functions, such as a stenting and a valving function, at a point of treatment within a body vessel, which may be desirable in certain situations, such as when a degree of vessel stenosis, occlusion, and/or weakening is present.

Support frames according to particular embodiments can include additional structural elements, such as additional struts and bends. The inclusion of additional struts and/or bends may be desirable, for example, in support frames and medical devices intended for implantation at locations in the body where lower radial force on the tissue is desired. For these embodiments, the inclusion of additional struts and/or bends can distribute the radial force of the support frame across more structural elements, thereby reducing the radial force exerted by a particular portion of the support frame against tissue at a point of treatment. A support frame according to an embodiment can include conventional structural features that facilitate anchoring of the support frame at a point of treatment within a body vessel, such as barbs and/or microbarbs, and structural features, such as radiopaque markers, that facilitate visualization of the support frame in conventional or other medical visualization techniques, such as radiography, fluoroscopy, and other techniques. Furthermore, a support frame according to an embodiment can include structural features, such as eyelets, barbs, fillets and other suitable structures, that provide attachment points for grafts and other materials.

In all embodiments, the support frame can be self-expandable or can require an input of force to affect expansion, such as a balloon expandable support frame. Each type of support frame has advantages and for any given application, one type may be more desirable than other types based on a variety of considerations. For example, in the peripheral vasculature, vessels are generally more compliant and typically experience dramatic changes in their cross-sectional shape during routine activity. Support frames and medical devices for implantation in the peripheral vasculature should retain a degree of flexibility to accommodate these changes of the vasculature. Accordingly, support frames and medical devices according to the invention intended for implantation in the peripheral vasculature, such as valve devices, advantageously include a self-expandable support frame.

In all embodiments, the support frames can be made from any suitable material and a skilled artisan will be able to select an appropriate material for use in a support frame according to a particular embodiment based on various considerations, including any desired flexibility and visualization characteristics. The material selected for a support frame according to a particular embodiment need only be biocompatible or be able to be made biocompatible. Examples of suitable materials include, without limitation, stainless steel, nickel titanium (NiTi) alloys, e.g., Nitinol, other shape memory and/or superelastic materials, molybdenum alloys, tantalum alloys, titanium alloys, precious metal alloys, nickel chromium alloys, cobalt chromium alloys, nickel cobalt chromium alloys, nickel cobalt chromium molybdenum alloys, nickel titanium chromium alloys, linear elastic Nitinol wires, polymeric materials, and composite materials. Also, absorbable and bioremodellable materials can be used. As used herein, the term “absorbable” refers to the ability of a material to degrade and to be absorbed into a tissue and/or body fluid upon contact with the tissue and/or body fluid. A number of absorbable materials are known in the art, and any suitable absorbable material can be used. Examples of suitable types of absorbable materials include absorbable homopolymers, copolymers, or blends of absorbable polymers. Specific examples of suitable absorbable materials include poly-alpha hydroxy acids such as polylactic acid, polylactide, polyglycolic acid (PGA), or polyglycolide; trimethlyene carbonate; polycaprolactone; poly-beta hydroxy acids such as polyhydroxybutyrate or polyhydroxyvalerate; or other polymers such as polyphosphazines, polyorganophosphazines, polyanhydrides, polyesteramides, polyorthoesters, polyethylene oxide, polyester-ethers (e.g., polydioxanone) or polyamino acids (e.g., poly-L-glutamic acid or poly-L-lysine). There are also a number of naturally derived absorbable polymers that may be suitable, including modified polysaccharides, such as cellulose, chitin, and dextran, and modified proteins, such as fibrin and casein.

Stainless steel and nitinol are currently considered desirable materials for use in the support frame due at least to their biocompatibility, shapeability, and well-characterized nature. Also, cold drawn cobalt chromium alloys, such as ASTM F562 and ASTM F1058 (commercial examples of which include MP35N™ and Elgiloy™, both of which are available from Fort Wayne Metals, Fort Wayne, IN; MP35N is a registered trademark of SPS Technologies, Inc. (Jenkintown, PA, USA); Elgiloy is a registered trademark of Combined Metals of Chicago LLC (Elk Grove Village, IL, USA)), are currently considered advantageous materials for the support frames at least because they are non-magnetic materials that provide beneficial magnetic resonance imaging (MRI) compatibility and avoid MRI artifacts typically associated with some other materials, such as stainless steel.

The support frames can be fabricated in any suitable manner and by any suitable technique. Skilled artisans will be able to select an appropriate manner and/or technique for fabricating a support frame according to a particular embodiment based on various considerations, including the nature of the material from which the support frame is being fabricated. Examples of suitable techniques include forming the support frame from wire, such as by wrapping a suitable wire around a suitable mandrel, by cutting the support frame from a tubular section of an appropriate material, such as by laser-cutting the support frame from a metal tubular member, and by forming the desired structure of the support frame in sheet form, such as by vapor deposition or other suitable technique, configuring the sheet into tubular form, such as by rolling or other suitable technique, and fixing the support frame in tubular form, such as by laser-welding or other suitable technique.

FIGS. 3 through 5 illustrate a second exemplary support frame 200.

The support frame 200 of this embodiment is similar to support frame 100 illustrated in FIGS. 1 and 2 and described above, except as detailed below. Thus, support frame 200 is an expandable support frame comprising proximal 202 and distal 204 portions connected by various connector segments 206, 208, 210, 212. The proximal portion 202 defines a first serpentine path 214 that extends around the circumference of the support frame 200. The distal portion 204 defines a second serpentine path 216 that also extends around the circumference of the support frame 200. The first serpentine path 214 includes straight strut portions 218 and bends 220. Each serpentine path 214, 216 is joined to connector segments 206, 208, 210, 212.

Similar to the first exemplary embodiment, connector segments 206 and 208 are disposed substantially opposite each other with respect to longitudinal axis a₁, and connector segments 210 and 212 are disposed substantially opposite each other with respect to longitudinal axis a₁. The support frame 200 includes only two connector segments 210, 212 that each include first and second struts, designated by the corresponding reference number along with a or b, e.g., 210a, 210b. Remaining connector segments 206, 208 each include only a single strut. This configuration is considered advantageous for support frames and medical devices in which a reduction in the overall amount of surface area of the support frame is desirable.

Also, the first 210a and second 210b struts of the first connector segment 210 are disposed at a slight angle with respect to each other and longitudinal axis a₁, placing the struts 210a, 210b in a skewed arrangement with respect to each other. A parallel or substantially parallel arrangement of the struts that comprise a particular connector segment is considered advantageous, but a skewed arrangement, such as the arrangement illustrated in FIG. 3, can be used if desired. In this embodiment, the first strut 210a defines first 250a and second 250b eyelets. Similarly, the second strut 212b defines first 252a and second 252b eyelets. Each of the eyelets 250a, 250b, 252a, 252b is a ring-shaped structure defining an opening. As best illustrated in FIG. 3, the first eyelets 250a, 252a are disposed on the struts 210a, 210b such that the center of each eyelet 250a, 252a is positioned on a transverse axis of the support frame 200 that intersects the connector segment 210 at a point that is about ¼^{th} of the height h₁ of the connector segment 210. The second eyelets 250b, 252b are disposed on the struts 210a, 210b such that the center of each eyelet 250b, 252b is positioned on a transverse axis of the support frame 200 that intersects the connector segment 210 at a point that is about ½ of the height h₁ of the connector segment 210. The inclusion of the eyelets 250a, 250b, 252a, 252b at these positions is considered advantageous at least because they provide attachment points at these positions for materials or additional elements included in medical devices that include the support frame 200, which can provide beneficial performance characteristics. If included, the eyelets can provide other and/or additional functional properties, also. For example, one or more eyelets can provide a structure for engagement by a suitable loading tool for placing a support frame or medical device within a delivery apparatus, such as a catheter. One or more eyelets can also be included to provide a structure for engagement by a suitable tool for withdrawing a support frame or medical device from a storage chamber, such as a hydration container within which a medical device is stored.

While the example support frame 200 includes four eyelets 250a, 250b, 252a, 252b, any suitable number of eyelets can be included in a support frame according to a particular embodiment. Furthermore, the each of the eyelets included in a support frame according to a particular embodiment can be placed at any suitable position on the connector segments for that support frame. Furthermore, the eyelet or eyelet on one straight strut in a connector segment can be positioned at the same or different position, relative to the height of the respective connector segment, as the eyelet or eyelets on another straight strut in a connector segment. A skilled artisan will be able to select an appropriate number of eyelets, an appropriate position for the eyelet or eyelets on the struts of a connector segment, and the relative distribution of the eyelet or eyelets on the straight struts of a connector segment in a support frame according to a particular embodiment based on various considerations, including any desired attachment points for an additional element, such as a graft or leaflet, that will be attached to the support frame, such as in the making of a medical device.

Also in this embodiment, the support frame 200 includes centering struts 244, 246, each of which extends in a proximal and radially outward direction from one of the straight strut portions 218 of the first serpentine path 214. The inventors have determined that the inclusion of centering struts 244, 246 provides beneficial deployment and positioning properties. For example, upon deployment in a body vessel, centering struts 244, 246 provide additional contact with the wall of the body vessel at the proximal portion 202 of the support frame 200, which can prevent or minimize tilting of the support frame 200 with respect to the longitudinal axes of the support frame 200 and the body vessel. If included, the centering struts can have any suitable size and configuration. For example, the centering struts can comprise straight struts, angled struts, a combination of straight struts and bends, as in the illustrated embodiment, or additional curvilinear struts. These struts, if included, can also provide a desirable location for placement of visualization makers, either as a structure fully or partially formed by these struts or as a structure attached to these struts.

In this embodiment, a series of connector struts 260, 262, 264, 266 extend between and join pairs of the connector segments 206, 208, 210, 212. Each of the connector struts 260, 262, 264, 266 extends between one of the connector segments 210, 212 that includes two struts, such as struts 210a and 210b, and one of the connector segments that includes only a single strut, such as connector segment 208. Thus, for example, connector strut 260 extends between and joins connector segments 210 and 206. Similarly, connector strut 262 extends between and joins connector segments 210 and 208.

Each of the connector struts 260, 262, 264, 266 lies on a plane that is disposed at an angle γ to a plane t₁ that orthogonally transects the longitudinal axis a₁ and includes the terminal structures of the distal portion 204 of the support frame 200. Each connector strut 260, 262, 264, 266 can lie on a plane disposed at any suitable angle. A skilled artisan will be able to determine an appropriate angle for each connector strut in a support frame according to a particular embodiment based on various considerations, including the nature of the body vessel within which the support frame is intended to be used, and the nature, size and configuration of any materials and/or additional elements that are attached to the support frame in the fabrication of a medical device that includes the support frame. Examples of suitable angles include angles between about 30° and about 50°, angles between about 30° and about 40°, and an angle that is about 35°.

While each of the connector struts 260, 262, 264, 266 in the illustrated embodiment is disposed at the same or substantially the same angle γ when the support frame is in its expanded configuration, different angles can be used for some or all of the connector struts. While considered advantageous, the illustrated configuration is merely an example of a suitable configuration.

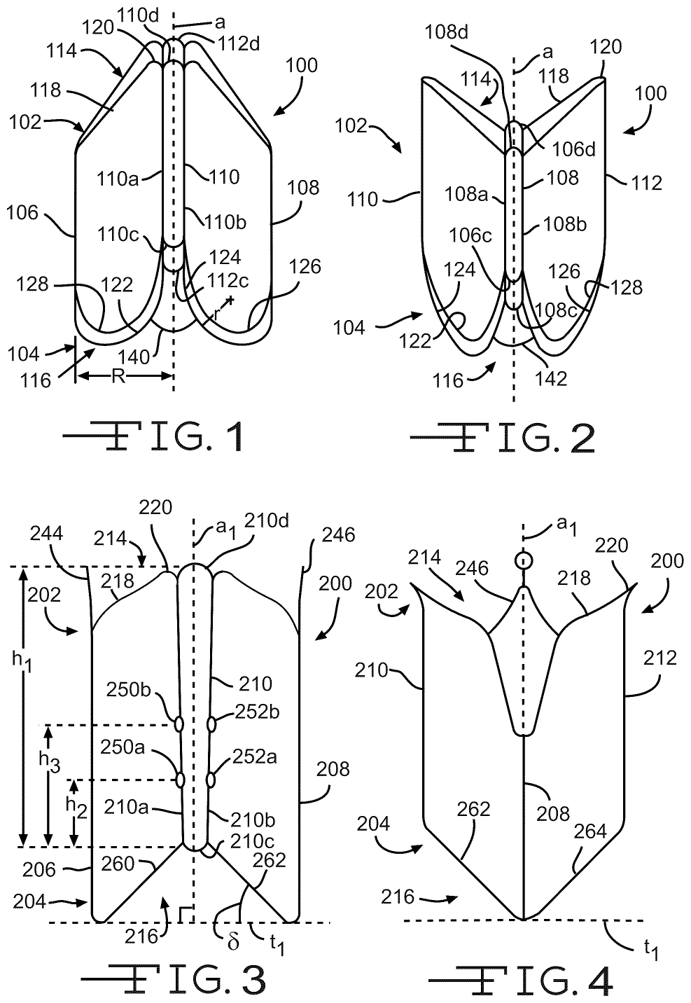

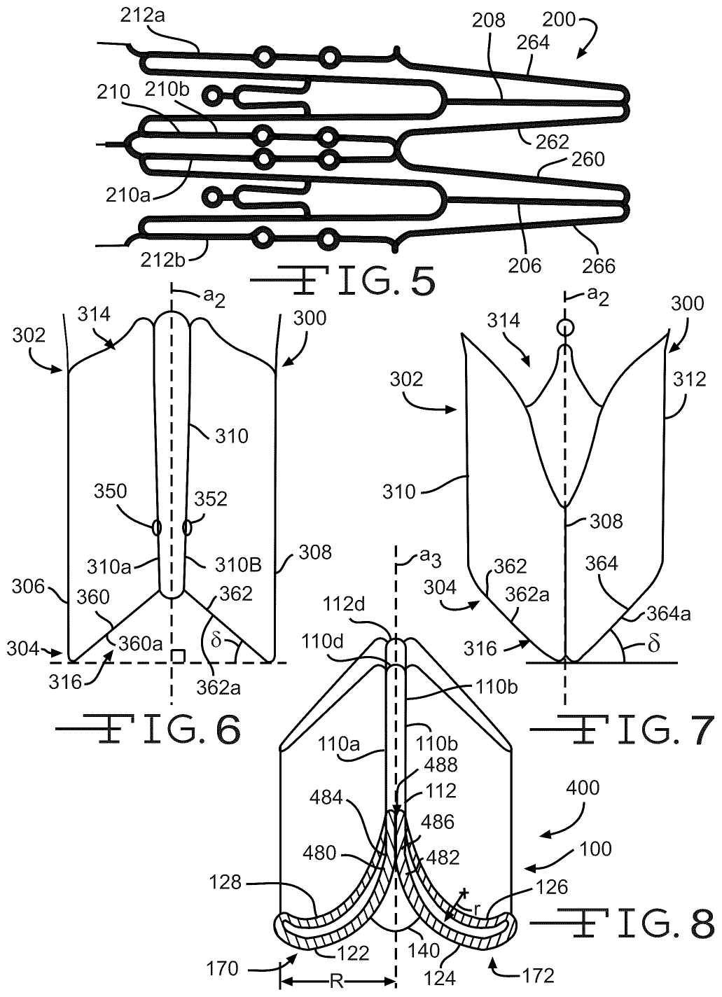

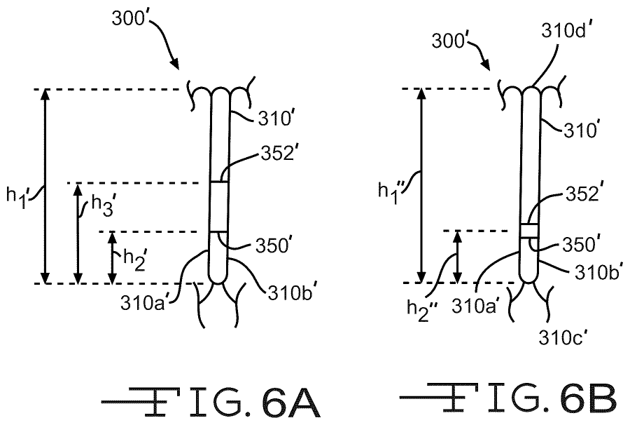

FIGS. 6 and 7 illustrate a third example support frame 300.

The support frame 300 of this embodiment is similar to support frame 200 illustrated in FIGS. 3 through 5 and described above, except as detailed below. Thus, support frame 300 is an expandable support frame comprising proximal 302 and distal 304 portions connected by various connector segments 306, 308, 310, 312. The proximal portion 302 defines a first serpentine path 314 that extends around the circumference of the support frame 300. The distal portion 304 defines a second serpentine path 316 that also extends around the circumference of the support frame 300. The first serpentine path 314 includes straight strut portions 318 and bends 320. Each serpentine path 314, 316 is joined to connector segments 306, 308, 310, 312.

Connector segments 306 and 308 are disposed substantially opposite each other with respect to longitudinal axis a₁, and connector segments 310 and 312 are disposed substantially opposite each other with respect to longitudinal axis a₁. Similar to the embodiment illustrated in FIGS. 3 through 5 and illustrated above, the support frame 300 includes only two connector segments 310, 312 that each include first and second struts, designated by the corresponding reference number along with a or b, e.g., 310a, 310b. Remaining connector segments 306, 308 each include only a single strut.

In this embodiment, the pair of struts that define each of connector segments 310 and 312 are disposed substantially parallel to each other. Also, each of the struts in the pair of struts that define each of connector segments 310 and 312 defines a single eyelet. Thus, as best illustrated in FIG. 6, the first strut 310a of connector segment 310 defines eyelet 350 and the second strut 310b defines eyelet 352. Each of the eyelets 350, 352 is a ring-shaped structure defining an opening. In this embodiment, each of the eyelets 350, 352 is disposed on the respective strut 310a, 310b such that the center of each eyelet 350, 352 is positioned on a transverse axis of the support frame 300 that orthogonally intersects the connector segment 310 at a point that is about ¼^{th} of the height h₁ of the connector segment 310. It is noted that, while the illustrated eyelets 350, 352 pass through the entire thickness of the respective struts 310a, 310b from one surface to an opposing surface, any other suitable structure can be used, such as passageways that pass through a partial thickness of the respective strut and/or blind openings.

In this embodiment, a series of connector struts 360, 362, 364, 366 extend between and join pairs of the connector segments 306, 308, 310, 312. Each of the connector struts 360, 362, 364, 366 extends between one of the connector segments 310, 312 that includes two struts, such as struts 310a and 310b, and one of the connector segments that includes only a single strut, such as connector segment 308. Thus, for example, connector strut 360 extends between and joins connector segments 310 and 306. Similarly, connector strut 362 extends between and joins connector segments 310 and 308.

In this embodiment, each of the connector struts 360, 362, 364, 366 is a curvilinear strut that includes a straight portion, designated by the corresponding reference number along with a. The straight portion 360a, 362a, 364a, 366a of each of the connector struts 360, 362, 364, 366 lies on a plane that is disposed at an angle γ to a plane that orthogonally transects the longitudinal axis a₁ and includes the terminal structures of the distal portion 304 of the support frame 300. Each connector strut 360, 362, 364, 366 can lie on a plane disposed with its respective straight portion 360a, 362a, 364a, 366a at any suitable angle. A skilled artisan will be able to determine an appropriate angle for each connector strut in a support frame according to a particular embodiment based on various considerations, including the nature of the body vessel within which the support frame is intended to be used, and the nature, size and configuration of any materials and/or additional elements that are attached to the support frame in the fabrication of a medical device that includes the support frame. Examples of suitable angles include angles between about 30° and about 50°, angles between about 30° and about 40°, and an angle that is about 35°.

While each of the connector struts 360, 362, 364, 366 in the illustrated embodiment is disposed with the respective straight portion 360a, 362a, 364a, 366a at the same or substantially the same angle γ when the support frame is in its expanded configuration, different angles can be used for some or all of the connector struts. While considered advantageous, the illustrated configuration is merely an example of a suitable configuration.

Each of FIGS. 6A and 6B illustrate a connector segment 310′ of an alternative support frame. In each figure, the connector segment 310′ includes alternative structure for the eyelets 350, 352 illustrated in FIG. 6. In these alternative embodiments, bars 350′, 352′ are included instead of the eyelets. The purpose of the bars 350′, 352′ is the same as the eyelets 350, 352 illustrated in FIG. 6, but the structure is different. Instead of defining an opening, each bar 350′, 352′ is a straight member or substantially straight member that extends between the pair of struts 310a′, 310b′ that define a connector segments 310′. If included in a support frame or medical device according to a particular embodiment, any suitable number of bars can be included and each of the included bars can be placed in any suitable position. In each of FIGS. 6A and 6B, two bars 350′, 352′ are included, but each figure illustrates a different relative positioning for the bars 350′, 352′.

If included, the bars 350′, 352′ can be positioned in a similar manner as the eyelets 350, 352 in the support frame 300 illustrated in FIG. 6. Thus, as illustrated in FIGS. 6A and 6B, one bar 350′ is disposed on the respective struts 310a′, 310b′ such that the lengthwise axis of the bar 350′ is positioned on a transverse axis of the support frame 300′ that orthogonally intersects the connector segment 310′ at a point that is about ¼^{th} of the height h₁′ of the connector segment 310′. This height is represented as h₂′ in FIGS. 6A and 6B.

Inclusion of additional bars is optional. If included, any additional bars can be positioned at any suitable location on the connector segment 310′. FIGS. 6A and 6B illustrate example positioning for a second bar 352′. In FIG. 6A, a second bar 352′ is disposed on the respective struts 310a′, 310b′ such that the lengthwise axis of the bar 352′ is positioned on a transverse axis of the support frame 300′ that orthogonally intersects the connector segment 310′ at a point that is about ½ of the height h₁′ of the connector segment 310′. This height is represented as h₃′ in FIGS. 6A. In this arrangement, the second bar 352′ is largely independent of the first bar 350′ and provides a second, independent point of attachment for additional materials, such as a valve leaflet or graft material.

In FIG. 6B, a second bar 352′ is disposed on the respective struts 310a′, 310b′ such that the bar is associated closely with the first bar 350′. In this embodiment, a hypothetical line extending between the pair of struts 310a′, 310b′ that define connector segment 310′ and that is spaced equidistantly from each of the bars 350′, 352′ is positioned on a transverse axis of the support frame 300′ that orthogonally intersects the connector segment 310′ at a point that is about ¼^{th} of the height h₁′ of the connector segment 310′. This height is represented as h₂′ in FIGS. 6A and 6B. In this arrangement, the second bar 352′ is paired with the first bar 350′ to cooperatively define an opening that provides a point of attachment for additional materials, such as a valve leaflet or graft material.

### Medical Devices

FIG. 8 illustrates a first exemplary medical device 400.

The medical device 400 is a valve device that includes the first example support frame 100 illustrated in FIGS. 1 and 2 and first 480 and second 482 leaflets. The first leaflet 480 is attached to the support frame 100 along a first attachment pathway 170 that extends along curvilinear struts 122, 128 and along a portion of connector segments 110, 112. Similarly, the second leaflet 482 is attached to the support frame 100 along a second attachment pathway 172 that extends along curvilinear struts 124, 126 and along a portion of connector segments 110, 112. Each leaflet 480, 482 has a free edge 484, 486 that is not attached to the support frame 100. The free edges 484, 486 cooperatively define valve orifice 488.

The medical device 400 is a valve device that is useful for regulating fluid flow through a body vessel. Each of the leaflets 480, 482 is movable between first and second positions. In the first position, the orifice 488 is open and allows fluid flow through the device 400 in a first direction. In the second position, the free edges 484, 486 of leaflets 480, 482 come together to close the orifice 488 and substantially prevent fluid flow through the device 400 in a second, opposite direction.

Each of the leaflets 480, 482 can have any suitable size, shape and/or configuration. A skilled artisan will be able to select leaflets having appropriate size, shape and configuration properties for a medical device according to a particular embodiment based on various considerations, including any desired performance characteristics of the medical device. The inventors have determined that leaflets that, when attached to a support frame and when the support frame is in an expanded configuration and the leaflets subjected to fluid pressure sufficient to effect closure of the valve orifice, define a domed radius of curvature, i.e., a portion of one surface of the leaflet lies on a portion of a spherical plane or substantially spherical plane, provide desirable performance characteristics for medical devices intended to be used as valve devices, such as prosthetic venous valve devices. In these embodiments, the portion of a spherical plane or substantially spherical plane can comprise a portion of the spherical plane of any suitable sphere. As a result, the portion of a spherical plane or substantially spherical plane can have any suitable radius of curvature. Also in these embodiments, the portion the surface of the leaflet that defines the domed radius can comprise any suitable portion of the leaflet surface, including a central portion that does not contact any struts or other structural members of the associated support frame, a base portion that is positioned at the bottom of a valve pocket formed in the valve device when the valve orifice is closed, or any other suitable portion of the leaflet surface. A skilled artisan will be able to select an appropriate portion of the leaflet surface and an appropriate radius of curvature for a medical device according to a particular embodiment based on various considerations, including the nature and size of the body vessel within which the medical device is to be implanted, and the nature of the material from which the leaflets are formed. Also, it is noted that the domed radii described herein are present in the respective leaflet at least when the leaflet is subjected to fluid pressure sufficient to close the associated valve orifice, such as when the medical device containing the leaflet is exposed to such fluid pressure in vivo or in suitable testing environments, such as in a vessel simulator or a simple fluid container.

The inventors have determined that leaflets defining a domed radius that is based on the radius of the circumference of the support frame in its expanded configuration provides desirable structural properties. For example, inclusion of one or more curvilinear leaflets that define a domed radius having a radius of curvature that is between about ⅛^{th} the radius of the circumference of the support frame in its expanded configuration and about 1× the radius of the circumference of the support frame in its expanded configuration is suitable. Additional examples of suitable radii of curvature include radii of curvature between about ¼^{th} and about ¾^{th} the radius of the circumference of the support frame in its expanded configuration, and a radius that is about ½ the radius of the circumference of the support frame in its expanded configuration.

The exemplary medical device 400 is illustrated with the support frame 100 in an expanded configuration and with the leaflets 480, 482 in the configuration they adopt when subjected to fluid pressure sufficient to effect closure of the valve orifice. As illustrated in the FIG., in this state, each of the leaflets 480, 482 defines a domed radius of curvature r that is about ½ the radius R of the circumference of the support frame 100 in its expanded configuration. The inventors have determined that this configuration of the leaflets 480, 482 is advantageous at least because of the beneficial performance characteristics provided by the arrangement.

It is noted that, while the medical device 400 is illustrated as including support frame 100, any suitable support frame that positions the leaflets 480, 482 in the desired configuration, i.e., with the domed radius, can be used. For example, leaflets can be attached to any of the support frame described and illustrated herein such that the desired configuration is achieved. A skilled artisan will be able to select an appropriate support frame for a medical device according to a particular embodiment based on various considerations, including the nature, size and configuration of the material forming the leaflets.

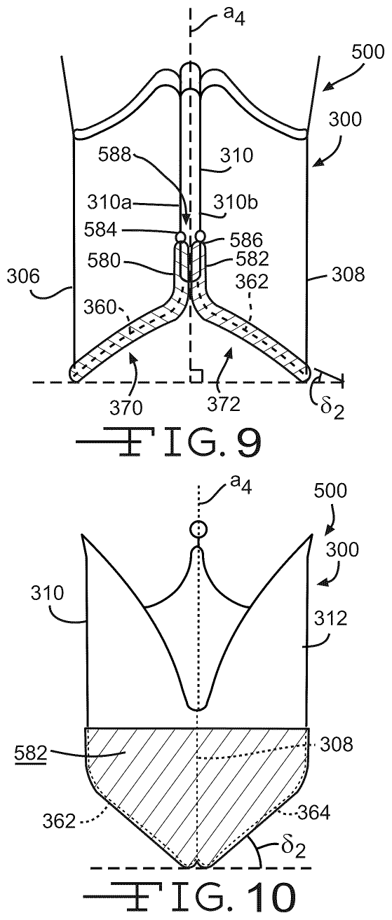

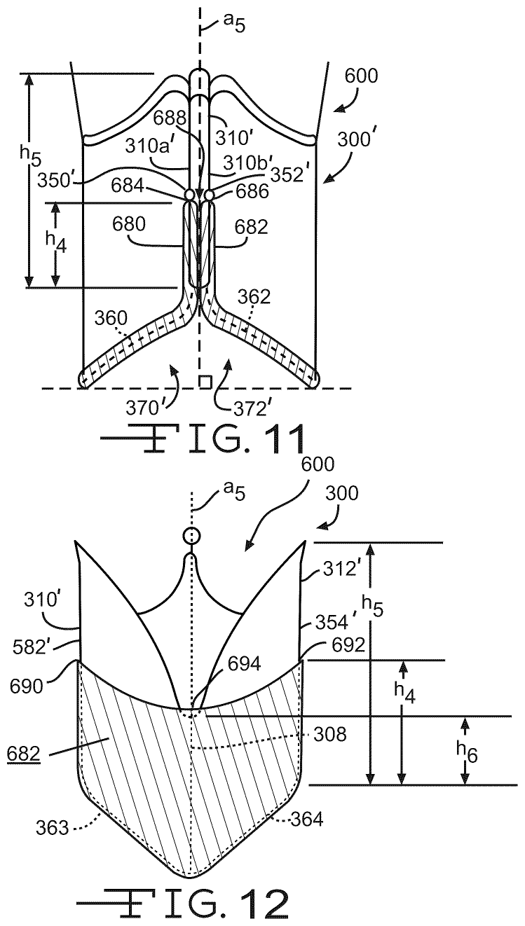

FIGS. 9 and 10 illustrate a second example medical device 500.

The medical device 500 is a valve device that includes the third exemplary support frame 300 illustrated in FIGS. 6 and 7 and first 580 and second 582 leaflets. The first leaflet 580 is attached to the support frame 300 along a first attachment pathway 370 that extends along connector struts 360, 366 and along a portion of connector segments 310, 312. Similarly, the second leaflet 582 is attached to the support frame 300 along a second attachment pathway 372 that extends along connector struts 362, 364 and along a portion of connector segments 310, 312. Each leaflet 580, 582 has a free edge 584, 586 that is not attached to the support frame 300. The free edges 584, 586 cooperatively define valve orifice 588.

The medical device 500 is a valve device that is useful for regulating fluid flow through a body vessel. Each of the leaflets 580, 582 is movable between first and second positions. In the first position, the orifice 588 is open and allows fluid flow through the device 500 in a first direction. In the second position, the free edges 584, 586 of leaflets 580, 582 come together to close the orifice 588 and substantially prevent fluid flow through the device 500 in a second, opposite direction.

In this embodiment, each attachment pathway 370, 372 extends along a portion of the axial length of connector segment 310 and along a portion of the axial length of connector segment 312. For each attachment pathway 370, 372 and each connector segment 310, 312, the portion of the axial length of the connector segment 310, 312 along which the attachment pathway extends can be any suitable portion of the axial length of the connector segment 310, 312, including the entire axial length of the connector segment 310, 312. For each attachment pathway and each connector segment in a medical device according to a particular embodiment, a skilled artisan will be able to select an appropriate portion of the axial length along which the attachment pathway extends based on various considerations, such as the nature, size and configuration of the leaflets or other material and/or additional elements included in the medical device.

The inventors have determined that a portion of the axial length of the connector segment along which the attachment pathway extends that is between about 1/16^{th} the full axial length of the connector segment and about the full axial length of the connector segment is suitable. Additional examples of suitable portions of the axial length of the connector segments along which the attachment pathways extend include portions of the axial length of the connector segments that are between about ⅛^{th} the full axial length of the connector segment and about ¾^{th} the full axial length of the connector segment, and portions of the axial length of the connector segments that are between about ¼^{th} the full axial length of the connector segment and about ½ the full axial length of the connector segment.

In the embodiment illustrated in FIGS. 9 and 10, each of the attachment pathways 370, 372 extends along a portion of each connector segment 310, 312 that is equal to about ¼^{th} the full axial length of the respective connector segment 310, 312. In this embodiment, the connector segments 310, 312 have approximately equal axial lengths, so the portions of the axial lengths along which the attachment pathways 370, 372 extend are also approximately equal. The inventors have determined that this configuration of the attachment pathways 370, 372 is advantageous for inclusion in medical devices according to particular embodiments at least because it provides desirable performance characteristics.

In any particular embodiment, the attachment pathways, if included, can extend along the same or different portion of the axial length of each connector segment. For example, a medical device according to an embodiment can include a first attachment pathway that extends along approximately equal portions of the axial lengths of first and second connector segments and a second attachment pathway that extends along approximately equal portions of the axial lengths of the first and second connector segments that are different than the portions along which the first attachment pathway extends. Furthermore, a medical device according to an embodiment may include one or more attachment pathways that extends along a portion of the axial length of a first connector segment and along a portion of the axial length of a second connector segment that is less than, equal to, approximately equal to, or greater than the portion of the axial length of the first connector segment.

In the illustrated embodiment, the attachment pathways 370, 372 also extend along connector struts 360, 362, 364, 366 that extend between and join adjacent pairs of connector segments 306, 308, 310, 312. If included, any suitable connector struts can be used in a medical device according to a particular embodiment and a skilled artisan will be able to select appropriate connector struts based on various considerations, including the nature of the material from which the element(s) being attached to the support frame, such as leaflets, is formed. As illustrated in FIGS. 9 and 10, connector struts 360, 362, 364, 366 that each comprise a curvilinear strut that includes a straight portion, designated by the corresponding reference number along with a, are considered suitable. In the illustrated embodiment, the straight portion 360a, 362a, 364a, 366a of each of the connector struts 360, 362, 364, 366 lies on a plane that is disposed at an angle γ₂ to a plane that orthogonally transects the longitudinal axis a₄ and includes the terminal structures of the distal portion 304 of the support frame 300. Each connector strut 360, 362, 364, 366 can be disposed with its respective straight portion 360a, 362a, 364a, 366a at any suitable angle. A skilled artisan will be able to determine an appropriate angle for each connector strut in a support frame according to a particular embodiment based on various considerations, including the nature of the body vessel within which the support frame is intended to be used, and the nature, size and configuration of any materials and/or additional elements that are attached to the support frame in the fabrication of a medical device that includes the support frame. Examples of suitable angles include angles between about 30° and about 50°, angles between about 30° and about 40°, and an angle that is about 35°.

FIGS. 11 and 12 illustrate a third example medical device 600.

The medical device 600 is a valve device that includes a modified version of the third exemplary support frame 300′ illustrated in FIGS. 6 and 7 and first 680 and second 682 leaflets. The medical device 600 is similar to the second exemplary medical device 500 described above and illustrated in FIGS. 9 and 10, except as detailed below. The first leaflet 680 is attached to the support frame 300′ along a first attachment pathway 370′ that extends along connector struts 360, 366 and along a portion of connector segments 310′, 312′. Similarly, the second leaflet 682 is attached to the support frame 300′ along a second attachment pathway 372′ that extends along connector struts 362, 364 and along a portion of connector segments 310′, 312′. Each leaflet 680, 682 has a free edge 684, 686 that is not attached to the support frame 300. The free edges 684, 686 cooperatively define valve orifice 688.

The medical device 600 is a valve device that is useful for regulating fluid flow through a body vessel. Each of the leaflets 680, 682 is movable between first and second positions. In the first position, the orifice 688 is open and allows fluid flow through the device 600 in a first direction. In the second position, the free edges 684, 686 of leaflets 680, 682 come together to close the orifice 688 and substantially prevent fluid flow through the device 600 in a second, opposite direction.

In this embodiment, support frame 300 includes eyelets 350′, 352′, 354′, 356′. The first 310a′ strut of the first connector segment 310′ defines eyelet 350′. Similarly, the second strut 310b′ of the first connector segment 310′ defines eyelet 354′. Similarly, the first 312a′ and second 312b′ struts of the second connector segment each defines one of remaining eyelets 354′, 356′. Each of the eyelets is a ring-shaped structure defining an opening. As best illustrated in FIG. 11, each of the 350′, 352′, 354′, 356′ is disposed on the respective strut 310a′, 310b′, 312a′, 312b′ of the respective connector segment 310′, 312′ such that the center of each eyelet 350′, 352′, 354′, 356′ is positioned on a transverse axis of the support frame 300′ that intersects the connector segments 310′, 312′ at a point corresponding to a height h₄ that is about ½ of the height h₅ of the respective connector segment 310′, 312′. The inclusion of the eyelets 350′, 352′, 354′, 356′ at these positions is considered advantageous at least because they provide attachment points at these positions for the leaflets 680, 682, which can provide beneficial performance characteristics.

In this embodiment, the free edge 684, 686 of each leaflet defines a curve. If leaflets having this structure are included, any suitable curve can be used and a skilled artisan will be able to select an appropriate curve or curves based on various considerations, including the nature of the material from which the leaflets are formed. As best illustrated in FIG. 12, a parabolic curve is considered suitable. Indeed, the inventors have determined that a parabolic curve that, when the respective leaflet 682 is attached to the support frame 300, extends inwardly from points 690, 692 on the respective attachment pathway 372 that correspond to a height h₄ is about ½ of the height h₅ of the respective connector segment 312′ to an apex 694 that is at a point that corresponds to a height h₆ that is is about ¼^{th} of the height h₅ of the respective connector segment 312′, is considered suitable.

If a leaflet having a free edge defining a curve is used, the curve can be formed prior to attaching the leaflet to the support frame, or can be formed following attachment of the leaflet to the support frame, such as by cutting the leaflet to create a free edge defining a desired curve.

In all embodiments, any suitable materials and/or additional elements can be attached to the support frame to form a medical device. A skilled artisan will be able to select an appropriate material for use with a support frame in a medical device according to a particular embodiment based on various considerations, including the intended use and desired function of the medical device. For valve devices, such as the valve device illustrated in FIGS. 9 and 10, each of the leaflets 580, 582 comprises a section of material, such as a sheet, that is attached to the support frame 300 along a respective attachment pathway 370, 372, as described above. The leaflets 580, 582 can be formed of any suitable material, and need only be biocompatible or be able to be made biocompatible. The material can advantageously be formed of a flexible material. Examples of suitable materials for use as leaflets in medical devices that comprise valve devices include natural materials, synthetic materials, and combinations of natural and synthetic materials. Examples of suitable natural materials include extracellular matrix (ECM) materials, such as small intestine submucosa (SIS), and other bioremodelable materials, such as bovine pericardium. Other examples of suitable ECM materials that can be used include stomach submucosa, liver basement membrane, urinary bladder submucosa, tissue mucosa, and dura mater. Other examples of suitable natural materials include renal capsule matrix, abdominal fascia, parenchyma, such as abdominal parenchyma, connective tissue, pulmonary or lung ligament, tissue laminates, and natural valve leaflets with or without adjacent vessel wall. Pleura is also considered a suitable natural material, including visceral pleura. Fixed tissues are also considered suitable, including fixed SIS, fixed pericardium, fixed pulmonary or lung ligament, and any other suitable fixed natural tissue. When fixed tissue is used, any suitable fixation technique and/or procedure can be used, including chemical fixatives, such as aldehydes, e.g., formaldehyde, gluteraldehyde, and formalin, and carbodiimides, such as ethyl dimethylaminopropyl carbodiimide, dicyclohexylcarbodiimide. Physical fixation techniques and/or procedures can also be used, including exposure to heat and/or radiation. Lyophilized preparations and chemically-dried preparations of these natural materials are also considered suitable. Examples of suitable synthetic materials include polymeric materials, such as expanded polytetrafluoroethylene, polyurethane, polyurethane urea, polycarbonate, and polyesters.

Any attached materials can have any suitable size, shape and configuration. For example, valve devices can include one, two or more leaflets that are sheet-like sections of material attached to a support frame according to an embodiment. Another example of a material that can be attached to a support frame according to an embodiment is a tubular structure that is attached around the outer circumference of the support frame. Indeed, a tubular structure and one, two or more leaflets can be attached to a support frame according to an embodiment to form a valve device having an outer sleeve.

In all embodiments including additional material and/or elements attached to the support frame, the additional material and/or elements can be attached to the support frame in any suitable manner and with any suitable structure and/or substance. For example, leaflets can be attached to a support frame in a valve device using sutures, tissue welding, adhesive(s), mechanical attachment(s), a combination of these approaches, and any other suitable structure and/or substance.

In all embodiments including an additional material and/or elements attached to the support frame, the additional material and/or elements can be attached to the support frame in any suitable orientation. A skilled artisan will be able to select a suitable orientation for a particular material or element attached to the support frame in a specific embodiment based on various considerations, including the physical properties of the material or element and any desired properties of the resulting medical device that may be impacted by the orientation of the material or element. For example, for valve devices that include one or more leaflets attached to the support frame, it may be desirable to attach the leaflet or leaflets in a particular orientation based on the ability of the leaflet to stretch in a particular direction. Anisotropic materials may be able to stretch to a greater degree along one axis than along another axis. The inventors have determined that, when attaching an anisotropic material to a support frame to form a medical device, it may be desirable to attach the material in an orientation in which the axis along which the material has a greater ability to stretch is aligned with the longitudinal axis of the support frame if it is desirable to have the leaflet of the medical device form a relatively deeper valve pocket when the medical device is subjected to sufficient fluid pressure to move the leaflet to a closed position. Conversely, the inventors have determined that it may be desirable to attach the material in an orientation in which the axis along which the material has a greater ability to stretch is aligned in a transverse orientation to the longitudinal axis of the support frame if it is desirable to have the leaflet of the medical device form a relatively larger valve orifice when the medical device is subjected to sufficient fluid pressure to move the leaflet to an open position.

For valve devices, the inventors have determined that attaching a leaflet to a support frame described herein while the leaflet is held in an open position can provide desirable performance characteristics to the resulting valve device. Specifically, the inventors have determined that attaching a leaflet to a support frame described herein while the leaflet is held in an open position on a mandrel such that a degree of slack is provided in a portion of the leaflet that will have a domed radius can provide desirable performance characteristics to the resulting valve device.

Furthermore, while the medical devices described and illustrated herein are valve devices, it is noted that other types of medical devices can be made in accordance with the disclosure. For example, a vessel occluder can include a support frame according to an embodiment along with leaflets that are sewn or otherwise attached to each other to permanently close an associated valve orifice or a graft material that lacks an orifice.

The support frames and medical devices can be implanted within a body vessel at a desired point of treatment using conventional minimally-invasive techniques, such as by delivery with an associated catheter, by surgical techniques, or by any other suitable technique for placing a support frame or medical device at a point of treatment within a body vessel.

The foregoing detailed description refers to example support frames and medical devices and includes the best mode for practicing the invention. The description and the appended drawings illustrating the described devices are intended only to provide examples and not to limit the scope of the claims in any manner.

What is claimed is:

1. A medical device for regulating fluid flow through a body vessel of a patient, comprising:
an expandable support frame having a longitudinal axis, an outer circumference, an unexpanded configuration, and an expanded configuration, the support frame comprising:
a proximal portion, a distal portion, a first connector segment, a second connector segment, a third connector segment, and a fourth connector segment, the proximal portion and the distal portion connected by the first, second, third, and fourth connector segments;
the proximal portion defining a first circumferential serpentine path joined to the first, second, third, and fourth connector segments;
the distal portion defining a second circumferential serpentine path joined to the first, second, third, and fourth connector segments and comprising a first connector strut extending between and joining the first and third connector segments, a second connector strut extending between and joining the second and third connector segments, a third connector strut extending between and joining the first and fourth connector segments, and a fourth connector strut extending between and joining the second and fourth connector segments;
the first connector segment having an axial length and comprising first and second struts and a first connecting bar extending between and joining the first and second struts, the first connecting bar positioned on a first transverse axis of the support frame that orthogonally intersects the first connector segment at a point between the distal portion and about ½ the axial length of the first connector segment;
the second connector segment disposed substantially opposite the first connector segment with respect to the longitudinal axis, the second connector segment comprising third and fourth struts and a second connecting bar extending between and joining the third and fourth struts;
the third connector segment disposed circumferentially adjacent the first and second connector segments, the third connector segment being only a single strut; and
the fourth connector segment disposed substantially opposite the third connector segment with respect to the longitudinal axis;
a first leaflet attached to the support frame along an attachment pathway extending along the first and second connector struts and along a first portion of the first connector segment and a first portion of the second connector segment, the first portion of the first connector segment extending from the distal portion, toward the proximal portion, and to the first connecting bar, the first portion of the second connector segment extending from the distal portion and toward the proximal portion, the first leaflet having a first free edge; and
a second leaflet attached to the support frame along a second attachment pathway extending along the third and fourth connector struts and along a second portion of the first connector segment and a second portion of the second connector segment, the second leaflet having a second free edge;
wherein the first and second free edges cooperatively define a valve orifice.

2. The medical device of claim 1, wherein the first transverse axis of the support frame orthogonally intersects the first connector segment at a point that is about ¼^{th} the axial length of the first connector segment.

3. The medical device of claim 1, wherein the second connector segment has an axial length; and
wherein the second connecting bar is positioned on a second transverse axis of the support frame that orthogonally intersects the second connector segment at a point between the distal portion and about ½ the axial length of the second connector segment.

4. The medical device of claim 3, wherein the second transverse axis of the support frame orthogonally intersects the second connector segment at a point that is about ¼^{th} the axial length of the second connector segment.

5. The medical device of claim 1, wherein the first connector segment has a third connecting bar extending between and joining the first and second struts.

6. The medical device of claim 5, wherein the third connecting bar is positioned on a third transverse axis of the support frame that orthogonally intersects the first connector segment at a point between the first connecting bar and the proximal portion.

7. The medical device of claim 5, wherein the second connector segment has a fourth connecting bar extending between and joining the third and fourth struts.

8. The medical device of claim 7, wherein the second connector segment has an axial length;
wherein the second connecting bar is positioned on a second transverse axis of the support frame that orthogonally intersects the second connector segment at a point between the distal portion and about ½ the axial length of the second connector segment; and
wherein the fourth connecting bar is positioned on a fourth transverse axis of the support frame that orthogonally intersects the second connector segment at a point between the second connecting bar and the proximal portion.

9. The medical device of claim 1, wherein the first and second struts of the first connector segment are substantially parallel; and
wherein the third and fourth struts of the second connector segment are substantially parallel.

10. The medical device of claim 1, wherein the fourth connector segment is only a single strut.

11. The medical device of claim 1, wherein each of the first connector strut, the second connector strut, the third connector strut, and the fourth connector strut comprise a curvilinear strut that includes a straight portion.

12. The medical device of claim 1, wherein the first portion of the first connector segment is between about ⅛^{th} and about ¾^{th} the axial length of the first connector segment;
wherein the second connector segment has an axial length; and
wherein the first portion of the second connector segment is between about ⅛^{th} and about ¾^{th} the axial length of the second connector segment.

13. The medical device of claim 1, wherein the first portion of the first connector segment is about ¼^{th} the axial length of the first connector segment;
wherein the second connector segment has an axial length; and
wherein the first portion of the second connector segment is about ¼^{th} the axial length of the second connector segment.

14. The medical device of claim 1, wherein the support frame is free of additional struts between the first connector segment and the third connector segment;
wherein the support frame is free of additional struts between the second connector segment and the third connector segment;
wherein the support frame is free of additional struts between the first connector segment and the fourth connector segment; and
wherein the support frame is free of additional struts between the second connector segment and the fourth connector segment.

15. The medical device of claim 1, wherein the first and second leaflets comprise a natural material.

16. The medical device of claim 1, wherein the first portion of the second connector segment extends from the distal portion, toward the proximal portion, and to the second connecting bar.

17. The medical device of claim 1, wherein the first leaflet is attached to the first connecting bar.

18. The medical device of claim 1, wherein the first leaflet is attached to the second connecting bar.

19. A medical device for regulating fluid flow through a body vessel of a patient, comprising:
an expandable support frame having a longitudinal axis, an outer circumference, an unexpanded configuration, and an expanded configuration, the support frame comprising:
a proximal portion, a distal portion, a first connector segment, a second connector segment, a third connector segment, and a fourth connector segment, the proximal portion and the distal portion connected by the first, second, third, and fourth connector segments;
the proximal portion defining a first circumferential serpentine path joined to the first, second, third, and fourth connector segments;
the distal portion defining a second circumferential serpentine path joined to the first, second, third, and fourth connector segments and comprising a first connector strut extending between and joining the first and third connector segments, a second connector strut extending between and joining the second and third connector segments, a third connector strut extending between and joining the first and fourth connector segments, and a fourth connector strut extending between and joining the second and fourth connector segments;
the first connector segment having an axial length and comprising first and second struts and first and second connecting bars, each of the first and second connecting bars extending between and joining the first and second struts, the first connecting bar positioned on a first transverse axis of the support frame that orthogonally intersects the first connector segment at a point between the distal portion and about ½ the axial length of the first connector segment, the second connecting bar positioned on a second transverse axis of the support frame that orthogonally intersects the first connector segment at a point between the first connecting bar and the proximal portion;
the second connector segment having an axial length and disposed substantially opposite the first connector segment with respect to the longitudinal axis, the second connector segment comprising third and fourth struts and third and fourth connecting bars, each of the third and fourth connecting bars extending between and joining the third and fourth struts, the third connecting bar positioned on a third transverse axis of the support frame that orthogonally intersects the second connector segment at a point between the distal portion and about ½ the axial length of the second connector segment, the fourth connecting bar positioned on a fourth transverse axis of the support frame that orthogonally intersects the second connector segment at a point between the third connecting bar and the proximal portion;
the third connector segment disposed circumferentially adjacent the first and second connector segments, the third connector segment being only a single strut; and
the fourth connector segment disposed substantially opposite the third connector segment with respect to the longitudinal axis;
a first leaflet attached to the support frame along an attachment pathway extending along the first and second connector struts and along a first portion of the first connector segment and a first portion of the second connector segment, the first portion of the first connector segment extending from the distal portion, toward the proximal portion, and to the first connecting bar, the first portion of the second connector segment extending from the distal portion, toward the proximal portion, and to the third connecting bar, the first leaflet having a first free edge; and
a second leaflet attached to the support frame along a second attachment pathway extending along the third and fourth connector struts and along a second portion of the first connector segment and a second portion of the second connector segment, the second leaflet having a second free edge;
wherein the first and second free edges cooperatively define a valve orifice.

20. A medical device for regulating fluid flow through a body vessel of a patient, comprising:
an expandable support frame having a longitudinal axis, an outer circumference, an unexpanded configuration, and an expanded configuration, the support frame comprising:
a proximal portion, a distal portion, a first connector segment, a second connector segment, a third connector segment, and a fourth connector segment, the proximal portion and the distal portion connected by the first, second, third, and fourth connector segments;
the proximal portion defining a first circumferential serpentine path joined to the first, second, third, and fourth connector segments;
the distal portion defining a second circumferential serpentine path joined to the first, second, third, and fourth connector segments and comprising a first connector strut extending between and joining the first and third connector segments, a second connector strut extending between and joining the second and third connector segments, a third connector strut extending between and joining the first and fourth connector segments, and a fourth connector strut extending between and joining the second and fourth connector segments;
the first connector segment having an axial length and comprising first and second substantially parallel struts and first and second connecting bars, each of the first and second connecting bars extending between and joining the first and second struts, the first connecting bar positioned on a first transverse axis of the support frame that orthogonally intersects the first connector segment at a point between the distal portion and about ½ the axial length of the first connector segment, the second connecting bar positioned on a second transverse axis of the support frame that orthogonally intersects the first connector segment at a point between the first connecting bar and the proximal portion;
the second connector segment having an axial length and disposed substantially opposite the first connector segment with respect to the longitudinal axis, the second connector segment comprising third and fourth substantially parallel struts and third and fourth connecting bars, each of the third and fourth connecting bars extending between and joining the third and fourth struts, the third connecting bar positioned on a third transverse axis of the support frame that orthogonally intersects the second connector segment at a point between the distal portion and about ½ the axial length of the second connector segment, the fourth connecting bar positioned on a fourth transverse axis of the support frame that orthogonally intersects the second connector segment at a point between the third connecting bar and the proximal portion;
the third connector segment disposed circumferentially adjacent the first and second connector segments, the third connector segment being only a single strut; and
the fourth connector segment disposed substantially opposite the third connector segment with respect to the longitudinal axis, the fourth connector segment being only a single strut;
a first leaflet attached to the support frame along an attachment pathway extending along the first and second connector struts and along a first portion of the first connector segment and a first portion of the second connector segment, the first portion of the first connector segment extending from the distal portion, toward the proximal portion, and to the first connecting bar, the first portion of the second connector segment extending from the distal portion, toward the proximal portion, and to the third connecting bar, the first leaflet having a first free edge, the first leaflet attached to the first connecting bar and the third connecting bar; and
a second leaflet attached to the support frame along a second attachment pathway extending along the third and fourth connector struts and along a second portion of the first connector segment and a second portion of the second connector segment, the second leaflet having a second free edge;
wherein the first and second free edges cooperatively define a valve orifice.
